(19) United States
(12) Patent Application Publication
(10) Pub. No.: US 2021/0093515 A1
(43) Pub. Date: Apr. 1, 2021

(54) SINGLE CLICK APP CONTROLLED POINT-OF-CARE PHARMACY DRUG DISPENSING

(71) Applicant: Accoy Pharmaceuticals, Inc., Palm Beach Gardens, FL (US)

(72) Inventor: Edmund L. Valentine, Palm Beach Gardens, FL (US)

(21) Appl. No.: 17/061,086

(22) Filed: Oct. 1, 2020

Related U.S. Application Data
(60) Provisional application No. 62/908,961, filed on Oct. 1, 2019.

(51) Int. Cl. A61J 7/04; G16H 20/13

(52) U.S. Cl. CPC A61J 7/0418; G16H 20/13

(57) ABSTRACT

A single action system and method for authorizing dispensing and thereafter dispensing a Drug dose from an App-Controlled Drug Dispenser. When the Dispense Button is activated by the Patient, it effectuates a Handshake, via Bluetooth or similar communications protocol, with the App. The App then receives the dispenser information including dispenser identification, Drug identifier, Drug expiration date, dispenser storage information, dispensing history, remaining doses, and dispenser alerts information, The App then confirms the requested dose is authorized by the prescription and ensures the medication has been stored within prescription parameters. Then it accesses and biometrically authenticates the digitally acquired Patient data required by the App's Dispensing Algorithm. The Drug Dispensing Algorithm then either authorizes the Drug Dispenser to dispense the dose or prevents dispensing the dose and generates a Patient alert. In response to the Single-Action selection of the Dispense Button, the dispenser's system utilizes its communication routine(s) to send the App a request to dispense the dose. The App receives the request and validates the Drug Dispenser and Drug with the Patient and the related data required by the App's Dispensing Algorithm. The Dispensing Algorithm then aggregates and analyses the data and makes a dispense or do not dispense the dose decision. When the Patient performs that Single-Action, the App stores the Patient Values, the Dispenser information, annotates the dose has been dispensed and the number of remaining doses, and the time data measurements were recorded and the time the dose was dispensed—enabling Point-of-Care Pharmacy.

### CROSS REFERENCE TO RELATED APPLICATIONS

This application claims priority from U.S. Provisional Patent Application Ser. No. 62/908,961 filed on Oct. 1, 2019. Furthermore, the contents of U.S. Provisional Patent Application No. 62/203,638 filed on Aug. 11, 2015, U.S. Provisional Patent Application No. 62/252,966 filed on Nov. 9, 2015, U.S. Provisional Patent Application No. 62/294,585 filed on Feb. 12, 2016, U.S. Provisional Patent Application No. 62/325,012 filed on Apr. 20, 2016, U.S. Provisional Patent Application No. 62/375,192 filed on Aug. 15, 2016, U.S. Provisional Patent Application No. 62/375,256 filed on Aug. 15, 2016, U.S. Provisional Patent Application No. 62/416,972 filed on Nov. 3, 2016, U.S. Provisional Patent Application No. 62/427,919 filed on Nov. 30, 2016, U.S. Provisional Patent Application No. 62/432,292 filed on Dec. 9, 2016, U.S. Provisional Patent Application No. 62/432,248 filed on Dec. 9, 2016, U.S. Provisional Patent Application No. 62/432,358 filed on Dec. 9, 2016, U.S. Provisional Patent Application No. 62/432,394 filed on Dec. 9, 2016, International Patent Application No. PCT/US16/46491 filed on Aug. 11, 2016, International Patent Application No. PCT/US2017/017665 filed on Feb. 13, 2017, and U.S. Provisional Patent Application No. 62/808,446 are incorporated herein by reference in their entirety to the full extent permitted by law.

### FIELD OF THE INVENTION

The present invention relates to a computer method and system for Single-click Dispensing of an App-controlled Drug Dispenser Drug dose, and more particularly, to a method and system for controlling the dispensing of a prescribed dose or precluding dispensing a prescribed dose if it does not comply with the prescription or if it may result in a dose-medicated adverse event.

### BACKGROUND OF THE INVENTION

There were 5.8 billion dispensed Prescriptions in 2017, up 1.7% over 2016 when adjusted for Prescription length. Adverse Drug events (ADEs) due to misuse pose substantial risk for Patients, including emergency department (ED) visits and hospitalizations. Each year in the United States, about 1 in every 250 Americans goes to a hospital emergency department because of an adverse Drug event. Many adverse Drug events are preventable. Misuse of Prescription Drugs means taking a Medication in a manner or dose other than prescribed. As an example, emergency department (ED) visits among children usually were due to ADEs caused by antibiotics and antipsychotics while three fourths of ADEs among older adults were caused by anticoagulants, antiplatelet agents, antidiabetic Drugs, and opioid analgesics. Misuse can also occur due to a Patient forgetting they took the Medication and then inadvertently taking another dose.

The societal cost of Prescription Drug ADEs are significant. As an example, an ED visit averages $6,000 and an opioid overdose hospitalization more than $92,400 (in 2015). The numbers are staggering, 1.4 million annual ED ADE visits cost more than $8.4 billion and the Prescription opioid overdose hospitalizations more than $3.7 billion—and this excludes non-hospital related medical costs.

### BRIEF SUMMARY OF THE INVENTION

An embodiment of the present invention describes a Single-click App-controlled Drug Dispenser method and system which enables one-click App-controlled Point-of-Care Pharmacy™ Drug dispensing. This means being able to click and dispense a prescribed Medication dose if the diagnostic screen, which works in the background, indicates the dose is within prescription parameters and that the dose will not result in a Drug mediated adverse event. The App and dispenser are integrated to ensure: 1) the Medication is only dispensed to or (in the case of a minor) for the Patient for whom the Drug is prescribed (avoid divergence); 2) that the Prescription is only dispensed per the Prescription instructions (preclude misuse); and 3) that the dispensing of each dose is controlled by the App's Dispensing Algorithm (e.g., ensuring prescription compliance and preventing dose mediated adverse events).

The present invention is able to use various Drug Dispenser configurations, designs, and sizes. They can be limited to a single Drug or configured to dispense multiple Drugs. The Drug Dispenser can be a disposable unit or a reusable unit. It can be a standalone unit with a resident App or a Tethered Drug Dispenser controlled by an App resident on an Interface Device (e.g., smartphone, computer, tablet, or similar devices or a combination thereof).

The respective dispenser dispenses solid, semisolid, or liquid Medications on a dose-by-dose basis—including but not limited to nasal sprays, microparticles, Tablets, buccal Tablets, sublingual Tablets, effervescent Tablets, chewable Tablets, Tablets, hard-shelled capsules, soft-shelled capsules, capsules, caplets, films, lozenges, pastilles, pills, granules, powders, oral solution, oral emulsion, oral suspension, syrup, suppositories, etc. They may be dispensed directly or using packaged units such as unit dose Blister Packs and Medication films foil packaged in individual units, etc.

When the Dispense Button on the Drug Dispenser is activated (e.g., by depressing the button or touching or tapping on a designated area on the dispenser or by voice command), the Drug dispensing sequence begins. The Drug Dispenser effectuates a Handshake with the App and then transmits the following information starting from the last time the information was uploaded to the App (i) current and historic dispenser temperatures and humidity levels, (ii) unsuccessful dispensing attempts, (iii) dispenser intrusion attempts, (iv) dispensing problem alerts, (v) date and time the previous dose or doses were dispensed, and (vi) number of remaining doses, etc.

Then the App automatically aggregates the required digitally captured physiological, psychological, lifestyle, concomitant Medications, and environmental data required by the Drug Dispensing Algorithm to make a dispense or do not dispense decision.

If the Algorithm allows dispensing, the App instructs the Drug Dispenser to dispense the dose.

If the Dispensing Algorithm ascertains: 1) that the Drug has expired, 2) that the Drug has not been properly stored, 3) that the dose is not authorized by the prescription, and/or 4) dispensing the dose would result in a dose mediated adverse event (ADR), then the dispenser will remain Locked, show an indicator light on the dispenser that the dose cannot be dispensed, display a message on the Interface Device (or on the standalone dispenser) as to why the Medication cannot be dispensed and send an alert (e.g., text, email, electronic medical record annotation, etc.) explaining why the Medication cannot be dispensed.

### BRIEF DESCRIPTION OF THE DRAWINGS

Advantages of embodiments of the present invention will be Apparent from the following detailed description of the exemplary embodiments thereof, which description should be considered in conjunction with the accompanying drawings in which:

FIG. 1 is an exemplary embodiment illustrating the logic flow for the method and system of Single Click App-controlled Point-of-Care Pharmacy Drug dispensing.

FIG. 2 is an exemplary embodiment of the logic flow when the Dispenser's Dispense Button is depressed/selected or voice activated.

FIG. 3 is an exemplary embodiment of the logic to effectuate a secure Handshake between the Drug Dispenser and the Interface Device (e.g., smartphone, tablet, computer, App on a standalone Dispenser, etc.).

FIG. 4 is an exemplary embodiment of the logic to authenticate the dispenser.

FIG. 5 is an exemplary embodiment of the logic to aggregate the digitally captured Patient data.

FIG. 6 is an exemplary embodiment of the logic when the Dispensing Algorithm makes a dispense or do not dispense the dose decision.

FIG. 7 is an exemplary embodiment of the logic when the dispenser receives the dispense or do not dispense the dose instructions from the App.

### DETAILED DESCRIPTION OF THE INVENTION

### I. Terms and Acronyms

Terms used in this document, AKA denotes terms used interchangeably:

Abuse (AKA Substance Abuse, Medication Abuse, Drug Abuse, Opioid Abuse) refers to the act or practice of improperly using Medications (examples include but are not limited to opioids, anxiolytics, antidepressants, stimulants). It also refers to the compulsive, excessive, and self-damaging use of habit-forming Drugs or of using opioids and other Medications in harmful amounts, leading to addiction or Dependence, serious physiological injury (such as damage to kidneys, liver, heart) and/or psychological harm (such as dysfunctional behavior patterns, hallucinations, memory loss), or death.

App Controlled refers to a Drug Dispenser whose dose dispensing is controlled by an App. The App may reside on an Interface Device or be resident on a standalone Drug Dispenser.

Biometric Authentication (AKA Biometric Identification, Biometric Sign On, Voice Print, Face Recognition, Iris Scan, Retinal Scan, Eye Scan, Palm Scan, Motion Pattern Recognition, Heart Rate Recognition, Heart Rhythm Recognition, Physiological Authentication, Fingerprint Scan, Log On Name, PIN Number, Authentication PIN, Two-Factor Authentication, 2FA, Multi-Factor Authentication, MFA) encompasses but is not limited to biometric technologies that digitally capture fingerprint, palm, full-hand, voice, facial, iris, retina, eye, motion pattern, heart rhythm, other unique physiological parameters used for authentication, and/or PIN numbers to limit access to the Patient. In this document the term also incorporates any system, while not biometric, that allows access via the use of two-factor (2FA) or multi-factor authentication (MFA) which may incorporate a Login Name in combination with a Password and/or any additional security information, a computer-generated password that is sent by a server via email and/or text message. Biometric Authentication also refers to the use of two or more of these biometric technologies in combination.

Compliance (AKA Adherence, Prescription Compliance, Medication Compliance, Drug Compliance) describes the degree to which a Patient correctly follows the Prescription's Drug dosing, dispensing and storage instructions.

Dispenser Button (AKA Single-Action Dispensing, Single-Action Drug Dispensing, On/Off Button, Biometric Button, Finger Scan Button, Single-click Dispenser Button, Single-Action Dispensing, Voice-Activated Dispensing) refers to a button or area on a Drug Dispenser which when depressed, touched, or tapped or voice activated will initiate the Drug Dispenser's dispensing routine. The switch and/or sensor on the Drug Dispenser may be used to turn the dispenser on and dispense each Drug dose. The App may control the power to the Dispenser Button and only make it available during certain authorized dispensing time windows. Dispensing may constitute different configurations—some examples include but are not limited to: 1) a simple on/off switch that is depressed by the Patient to dispense a Dose when the App has authorized the Drug Dispenser to dispense the dose, 2) a Biometric on/off switch that dispenses a Dose when the App authorized dose dispensing upon Patient authentication, 3) a Biometric Authentication device that authenticates the Patient and then dispenses the authorized dose, 4) a Dispenser Button where the Firmware is programmed to allow the Patient to dispense a number of doses between Patient App interfaces, 5) a Dispenser Button where the Firmware is programmed to allow the Patient to dispense a number of doses, in keeping with Prescription intervals, between Patient App interfaces, 6) a Dispenser Button where the Firmware checks to see if an Interface Device is connected before dispensing a dose that is authorized to be dispensed by the prescription and to be dispensed without Biometric authentication and screening by the App. This may or may not require integrated Biometric Authentication. Each may be voice activated.

Dispensing Algorithm (AKA Algorithm) refers to the list of detailed instructions to effectuate a Handshake with digital devices, biometrically authenticate the device as being one that is aggregating the desired Patient's data, aggregate and analyze the information, and then use the decision logic to make a dispense or do not dispense decision. If it is a do not dispense decision, then the logic to inform the Patient of the decision and what steps the Patient will need to take to get the dose.

Diastolic Blood Pressure (AKA DBP) refers to the pressure the blood exerts within the arteries in between heartbeats, that is, when the heart is not actively ejecting blood into the arteries. After the heart is finished contracting, the cardiac ventricles relax momentarily so that they can be refilled with blood, in preparation for the next contraction. This period of ventricular relaxation is called “diastole,” and the blood pressure during diastole is called the Diastolic Blood Pressure.

Dose-by-Dose Dispensing (AKA Dose Dispensing) refers to the Drug Dispenser's ability to control dose dispensing as well as the ability of the tethered App to be able to exercise dose dispensing control on a dose-by-dose basis.

Drug (AKA pharmaceutical, Medication, medicament, OTC Drug, supplement, or herbal remedy)

Drug Dispenser (AKA Point of Care App Controlled Drug Dispenser, Disposable Point of Care App Controlled Drug Dispenser, Disposable Drug Dispenser, Dispensing Device, Standalone Dispenser, Dispenser Unit, Dispensing Unit, Reusable Drug Dispenser, Refillable Drug Dispenser, Drug Dispensing Unit, Tethered Drug Dispenser) refers to the disposable or reusable Drug Dispensing Unit whose dispensing is controlled by a Drug Specific App or a Standalone Drug Dispenser with a resident Drug Specific App or a Drug dispensing program that controls dose Drug dispensing.

Handshake (AKA Digital Handshake) refers to an exchange of signals between devices ensuring synchronization whenever a connection, as with another device, is initially established.

Heart Rate (AKA Heart Rhythm, HR) refers to the number of times the heart beats in a minute. This is the number of times it pumps to push blood round the body. The Heart Rhythm is the pattern in which the heart beats. Both are unique to each individual and may be used to biometrically authenticate the Patient.

Interface Device refers to the smartphone, tablet, computer, or standalone Drug Dispenser, or similar device with Bluetooth and/or Internet communications capabilities or like communications capabilities where the App resides.

Locked indicates the Drug cannot be dispensed by the Drug Dispenser until the Medication Specific App or the standalone device's dispensing software unlocks the Drug Dispenser and allows it to dispense the Medication.

Medication (AKA Drug, Pharmaceutical) refers to a substance used for medical treatment, specifically a medicine, legal or illegal Drugs, OTC Medications, vitamins, dietary supplements, herbal medicines, and/or recreational Drugs.

Patient (AKA Person) refers to the individual that is prescribed one or more Medications and/or is taking one or more Medications.

Patient and Drug Specific App (AKA Patient and Medication Specific App, Patient and Drug Specific App, Drug Specific App, Drug and Patient Specific App, Medication Specific App, Patient Specific App) refers to an App used to control Drug Dispenser dose dispensing. The App restricts certain information, such as prescription information, to only be changed by authorized personnel. It also contains Routines that restrict: 1) utilization by the Patient or authorized care giver, 2) dispensing to specific one or more Tethered Drug Dispensers that contain the prescribed Drug, 3) Dose-by-Dose Dispensing to only doses authorized by the Dispensing Algorithm.

Point-of-Care Pharmacy refers to a system comprised of a: 1) Medication, 2) Patient and Drug Specific App, 3) Drug Dispenser, 4) 24 hours per day and 365 days per year App, Drug Dispenser, and Medication support, 5) related data analytics, and 6) a Patient electronic medical record (EMR). It is designed to ensure: 1) the dispensed Medication is effective for the Patient, 2) only dispensed per the Prescription, 3) uses a Dispensing Algorithm which utilizes the Patient's digitally captured and/or Patient tested/reported/entered (Patient Entered) physiological, psychological, lifestyle, environmental, and/or concomitant Medication information to control Drug dose dispensing, 4) captures key point-of-care information to enable Medication management and better Patient and disease/condition management. It also refers to the ability of the system to prevent dispensing a prescribed dose, even it would otherwise be allowed by the prescription, if the digitally captured and/or Patient entered data indicate the Patient may suffer a Medication mediated adverse event if the Patient takes the dose.

Prescriber is defined as any healthcare professional authorized by an individual country or state to write a Prescription for a Drug. Examples include but are not limited to physicians, physician assistants, nurse practitioners, nurses, pharmacists, medics, etc.

Prescription (AKA ePrescription, Paper Prescription, Manually Entered Prescription, Digital Prescription, Drug Prescription, Medication Prescription) is an instruction electronically entered or manually written by an authorized medical practitioner that authorizes a Patient to be provided a medicine or treatment.

Routines (AKA Software Program, Software Routines, Subroutine, Procedure, Function, Method, Subprogram) is a portion of software code within a larger program that performs a specific task and is relatively independent of the remaining code.

Serial Number (AKA Unique Device Identifier, UDI, Serial No., SN, S/N, Identification Number, Tracking Number, Identifier) is a unique number or letters and numbers used for identification, tracking, and/or inventory purposes.

Single Click (AKA Single Action) refers to a patient taking a single action to cause a drug dispenser to dispense a controlled medication dose. That selection may be done by clicking, tapping, depressing, and/or touching on a Dispense Button on the drug dispenser to dispense a prescription authorized dose. This can also be effectuated via voice selection.

Smartwatch (AKA Wearable Sensor) as used in this document refers to any external device to which the App can tether to measure Patient values or capture Patient information that is required by a Drug and Patient Specific App to control Dose-by-Dose Dispensing from a Tethered Drug Dispenser.

Systolic Blood Pressure (AKA SBP) refers to The pressure exerted by blood flowing through the arteries. It is not constant but is dynamic, and constantly reflects what the heart is doing at a given moment. When the heart is actively beating (an event called “systole”), it is ejecting blood out into the arteries. This dynamic ejection of blood into the arteries causes the pressure within the arteries to rise. The peak blood pressure reached during active cardiac contraction is called the Systolic Blood Pressure.

Tablets as used in this invention refers to all types of solid, semisolid, or liquid oral dose Medications delivered on a Dose-by-Dose basis and in all the various shapes and sizes and unit dose containers. This includes but is not limited to Tablets, buccal Tablets, sublingual Tablets, effervescent Tablets, chewable Tablets, Tablets, hard-shelled capsules, soft-shelled capsules, capsules, caplets, films, lozenges, pastilles, pills, gel caps, granules, powders, microparticles, oral solution, oral emulsion, oral suspension, syrup, suppositories, Medication strips, nasal sprays, etc. They may be dispensed directly or dispensed using packaged units (such as unit dose Blister Packs) or Medication films foil packaged in individual Medication strip units, etc.

Tethered Drug Dispenser is a Drug Dispenser that utilizes the Interface Device's capabilities to offer functionality and ease of use that would not otherwise be possible in a Standalone Drug Dispenser with the same outside dimensions, the same size, or a battery life equal to or greater than the Medication's shelf life. It also refers to a Drug Dispenser that cannot function without being tethered to an App.

### II. List of Medications the Invention in its Various Embodiments is Applicable to

The invention and its various embodiments can enable better dose dispensing control, decreasing misuse, Diversion and accidental ingestion. This improves the Drug's safety profile and should decrease Drug mediated healthcare professional interventions, emergency department (ED) visits, and hospitalizations.

The invention is Applicable to all Tablets, as defined, that are marketed or in development. Drug compounds of interest are listed in: Goodman & Gilman's, The Pharmacological Basis of Therapeutics (13th Ed) (Goodman et al. eds) (McGraw-Hill) (2018); and latest mobilePDR (formerly Physician's Desk Reference); Cortellis Clinical Trials Intelligence databases by Clarivate Analytics; Adis Insight databases by Springer; and/or Pharmaprojects databases by Citeline, and pipeline and commercially marketed Medications contained in databases by IQVIA. These Drugs are encompassed in the embodiment of the invention by reference.

### III. Single-Click App-Controlled Point-of-Care Pharmacy Drug Dispensing

Various embodiments will be described hereinafter with reference to the accompanying drawings. These embodiments are illustrated and described by example only and are not intended to be limiting. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration”. Any embodiment described herein as “exemplary” or “example” is not necessarily to be construed as preferred or advantageous over other embodiments. Likewise, the term “embodiments of the invention” does not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., Application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The present invention provides a method and System for Single-Action Drug Dispensing in an App/App Controlled Drug Dispenser environment. The Single-Action Drug Dispensing System of the present invention reduces the number of Patient interactions needed to Dispense an App-controlled Medication dose. In one embodiment, the Drug Dispenser has a unique identifier which is used to limit dispensing control to authorized Tethered Drug and Patient Specific App(s). The App stores sensitive Patient and prescription information, aggregates digital Patient data required by the Dispensing Algorithm, and Biometrically authenticates the Drug Dispenser as well as each device where the App effectuates a Handshake to download Patient Biometrically Authenticated Data. The Dispensing Algorithm may utilize one or more single point in time data or trended data to make the dispense or do not dispense the dose decision. When the Patient wants to dispense a prescribed dose, the Patient activates the Dispenser Button on the Drug Dispenser to Send a request to the App on an Interface Device for authority to dispense the dose. The App determines whether the Patient identifier is mapped to the Drug Dispenser and the Drug and the Tablet strength contained in the Drug Dispenser. If so mapped, it then ensures the dose is authorized by the Prescription, the Drug has been stored in the Drug Dispenser per the Prescription, and if yes, proceeds to Handshake with one or more devices or Apps where data required by the Dispensing Algorithm are measured and/or stored. The Dispensing Algorithm then analyses the data and uses its decision tree to determine if the Single-Action Drug Dispensing is enabled. If yes, the App sends a signal to the Drug Dispenser to Dispense the dose. When Single-Action Drug Dispensing is enabled, the Patient need only perform a Single-Action (e.g., click or touch the Dispense Button on the Drug Dispenser) to Dispense the dose. When the Patient performs that Single-Action, the App stores the Patient Values, the Dispenser information, annotates the time the dose was dispensed and the number of remaining doses, and records the time stamped data measurements/values. Then the App updates the cloud-based Patient files related the Patient's Electronic Medical Record. Thus, when the App alerts the Patient a dose is due or when the Patient chooses to take a dose by selecting the Dispense Button, it only takes a Single-Action, i.e., depress or select the Dispenser Button, to capture required Point-of-Care Pharmacy data and dispense the dose. Also, since Patient sensitive information is already securely stored on the App and the App uses Biometric Authentication to ascertain the digitally captured Patient Data belongs to the Patient and the Drug Dispenser is tethered to the App, there is no need to reenter sensitive, protected Patient information. If the App's Dispensing Algorithm ascertains dispensing the prescribed dose may result in a Medication dose mediated adverse event, the Drug Dispenser remains Locked, an alert is highlighted on the Drug Dispenser, and Patient instructions regarding next steps is presented on the Interface Device screen.

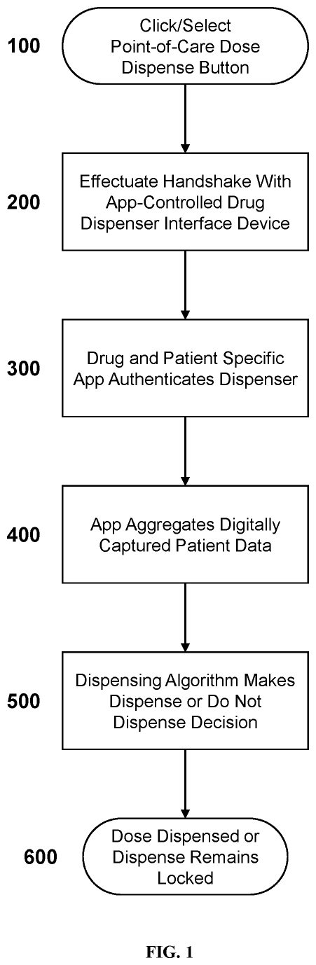

FIG. 1 illustrates an exemplary embodiment of the invention and logic effectuated when the Dispenser Button is selected. The process begins with clicking on or selecting the Dispense Button 100. This results in the Drug Dispenser's firmware effectuating a Handshake with the App on the App-Controlled Drug Dispenser resident on the Interface Device 200. The App then authenticates the Tethered Dispenser 300. The App then effectuates Handshakes with the Appropriate data aggregation devices and/or Apps and aggregates the Dispensing Algorithm required data 400. The Dispensing Algorithm then evaluates the data and makes a dispense or do not dispense decision 500. The dose is then either dispensed or the Drug Dispenser remains Locked 600.

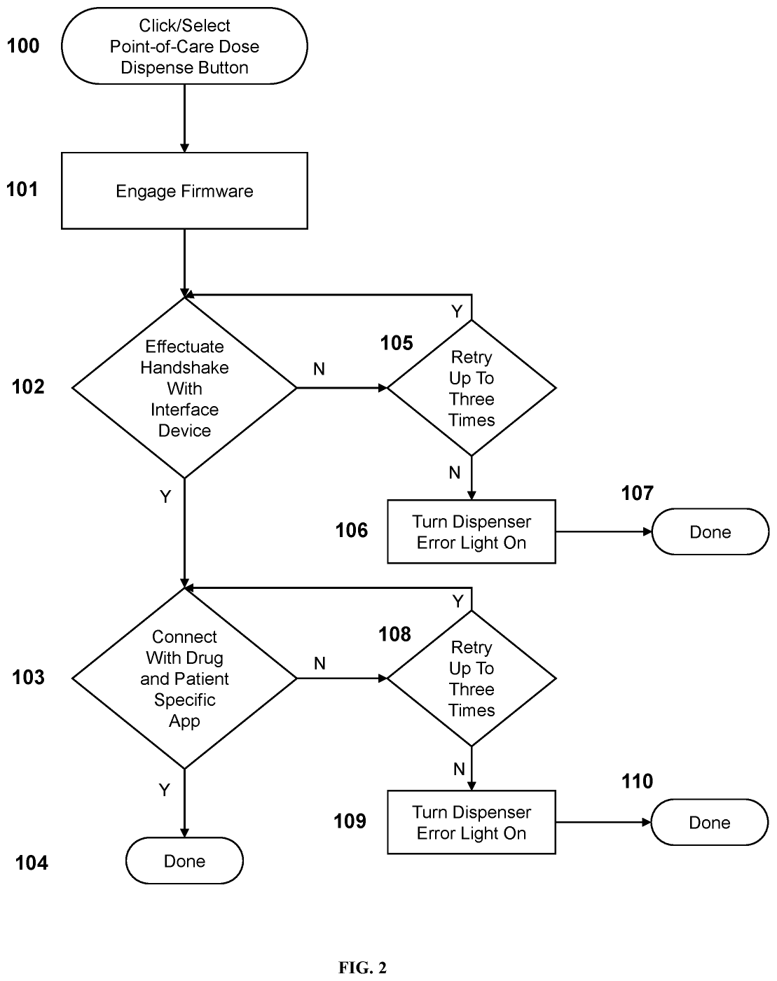

FIG. 2 illustrates an exemplary embodiment of the logic effectuated when the Dispense Button is clicked or selected 100. When the Dispense Button is clicked or selected 100, it wakes up and engages the Firmware 101. The Firmware then launches a Routine to effectuate a Handshake with the Interface Device 102. If it cannot effectuate the Handshake, it will try again up to three times 105. If it cannot effectuate the Handshake after three attempts, it turns on the Dispenser error light 106 at which point the logic is done 107 making further Handshake attempts. If it successfully effectuates a Handshake with the Interface Device 102, the Firmware then launches a Routine to connect with the Drug and Patient Specific App 103. If it cannot effectuate the connection, it will try again up to three times 108. If it cannot effectuate the connection after three attempts, it turns on the Dispenser error light 109 at which point the logic is done 110 making further Handshake attempts. If the Routine connects with the Drug and Patient Specific App 103, then it is done 104 and the App moves to the authentication Routine 200. Any failures to connect 107, 110 are recorded and retained in Firmware memory to assist in troubleshooting.

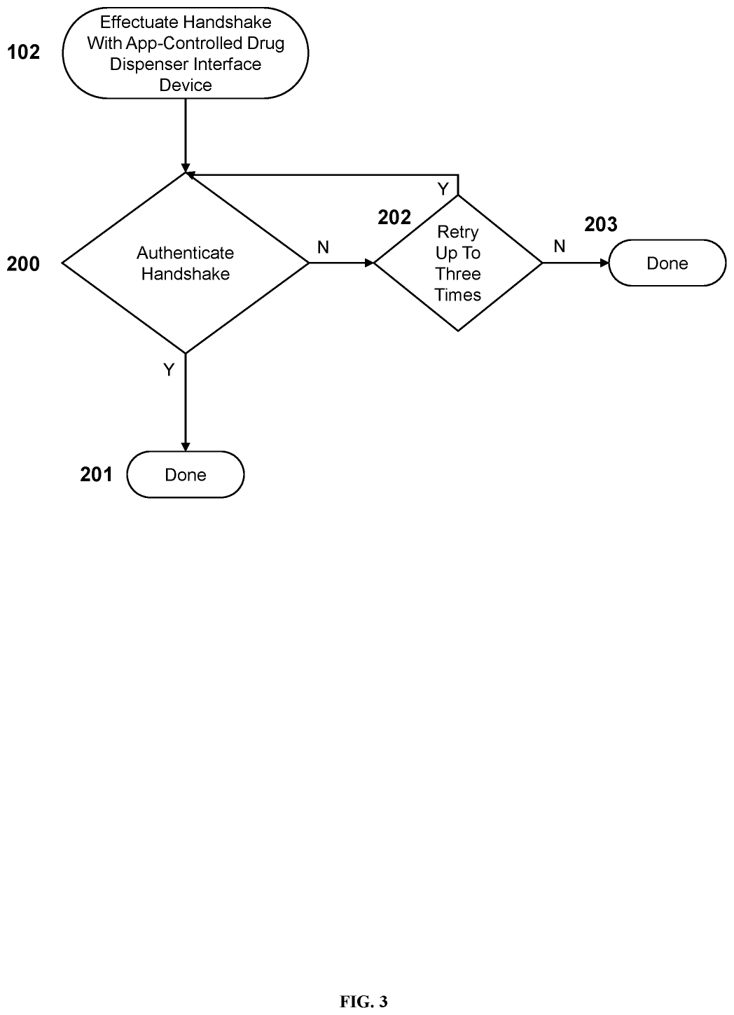

FIG. 3 illustrates an exemplary embodiment of the logic to authenticate the Handshake 200. When the Drug Dispenser attempts to effectuate a Handshake with the App-Controlled Drug Dispenser Interface Device 102, it tries to Biometrically Authenticates the Handshake 200. If it is unable to authenticate the Handshake, it will try up to three times 202. If after three attempts, the Biometric Authentication routine is unable to authenticate the Handshake, the Routine is done 203. This means the attempt to effectuate the Handshake with the Interface Device 102 failed. If the Handshake is authenticated 200, then the routine is done 201 and the App moves to the Drug Dispenser authentication Routine 300. Any failures to connect 203 are recorded and retained in Firmware memory to assists in troubleshooting.

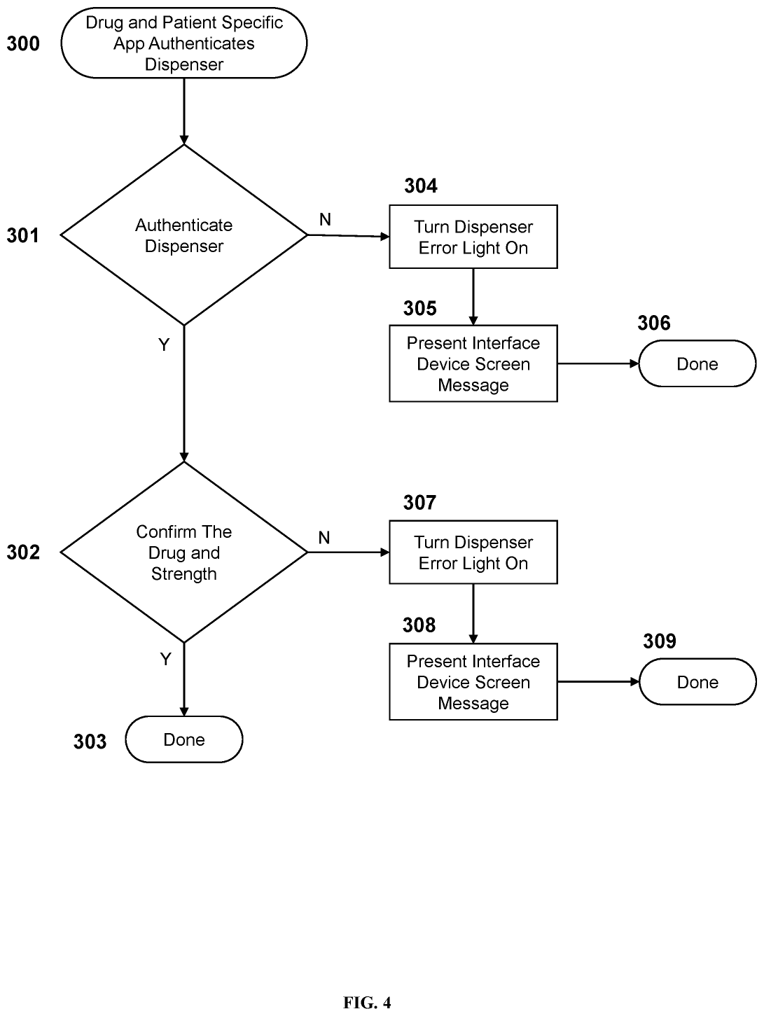

FIG. 4 illustrates an exemplary embodiment of the logic to authenticate the Drug Dispenser 300. When the Drug and Patient Specific App 300 tries to authenticate the dispenser 301, it ensures the Serial Number for the Tethered Drug Dispenser matches the related Serial Number in the App. If it does not match, it turns the Drug Dispenser's error light on 304, presents a message on the Interface Device screen with instructions on how to resolve the problem 305, and then stops the authentication Routine 306. If the Drug Dispenser is authenticated 301, the App confirms the Drug and dosage strength contained in the Drug Dispenser match the Prescription 302. If the Drug and/or strength do not match the prescribed Drug and strength, the Routine turns on the Drug Dispenser's error light 307 presents a message on the Interface Device screen with instructions on how to resolve the problem 308, and then stops the authentication Routine 309. This leaves the Drug Dispenser Locked. If the Drug and strength match the prescription 302, then the Routine is done 303 and the App moves to the data acquisition and aggregation Routine 400. Any failures to authenticate 306, 309 are recorded and retained in Firmware memory to assists in troubleshooting.

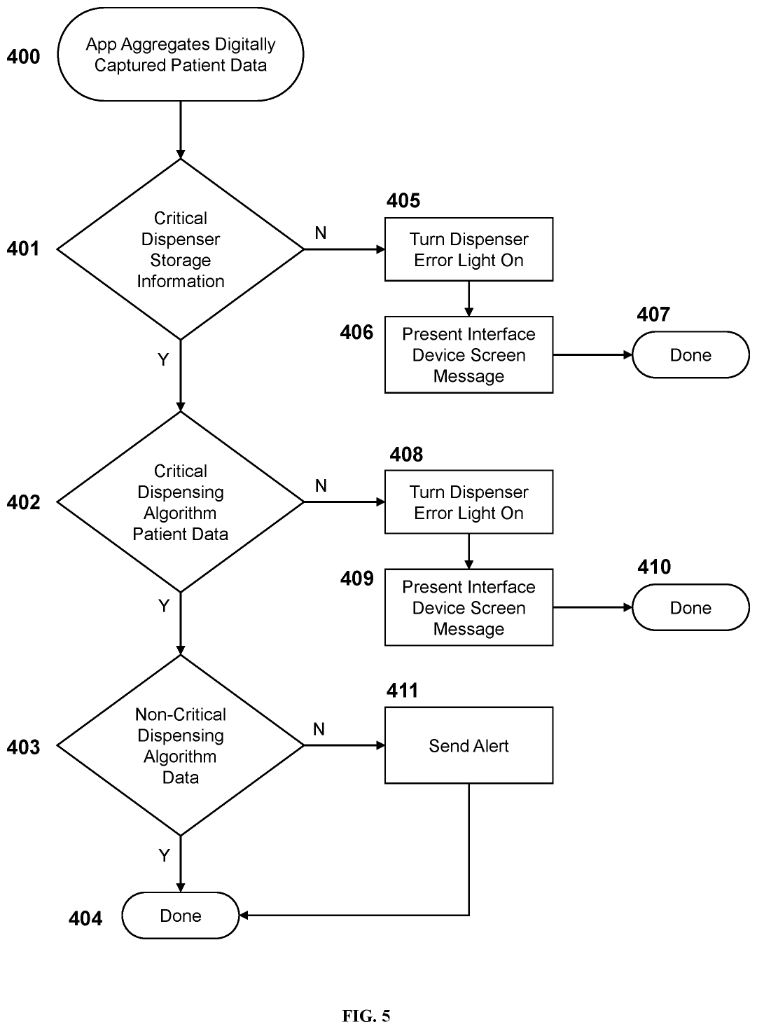

FIG. 5 illustrates an exemplary embodiment of the logic for the App to aggregate digitally captured Patient data 400. The App begins by checking to ensure the dose is authorized by the prescription and if the Medication 401 has been stored per the prescription requirements (e.g., heat and humidity levels). If the values are not in compliance, the Routine will turn on the Drug Dispenser's error light 405 and present Patient instructions on the Interface Device 406 on what the Patient has to do to get a refill prescription. At that point the Routine is done 407. If the Medication has been stored correctly 401, it will ascertain what information is required for the Dispensing Algorithm 402. Then it will proceed to make the requisite Handshakes with the respective devices to aggregate the required data/values. If the Drug Dispenser is unable to effectuate the required Handshake and/or if the required data is not available, the Routine will turn on the Drug Dispenser's error light 408 and present Patient instructions on the Interface Device 409, then the Routine will be done 410. After aggregating the required Dispensing Algorithm data 402, the Routine will effectuate Handshakes, as required, to aggregate non-critical Drug Dispensing Algorithm data 403. If a Handshake cannot be effectuated and/or if the data is not available, the Routine will send an alert indicating the information was not available and what the Patient needs to do to make it available 411. At this point the Routine is done 404 and the App proceeds to analyze the aggregated data to make a dispense or do not dispense the dose decision 500.

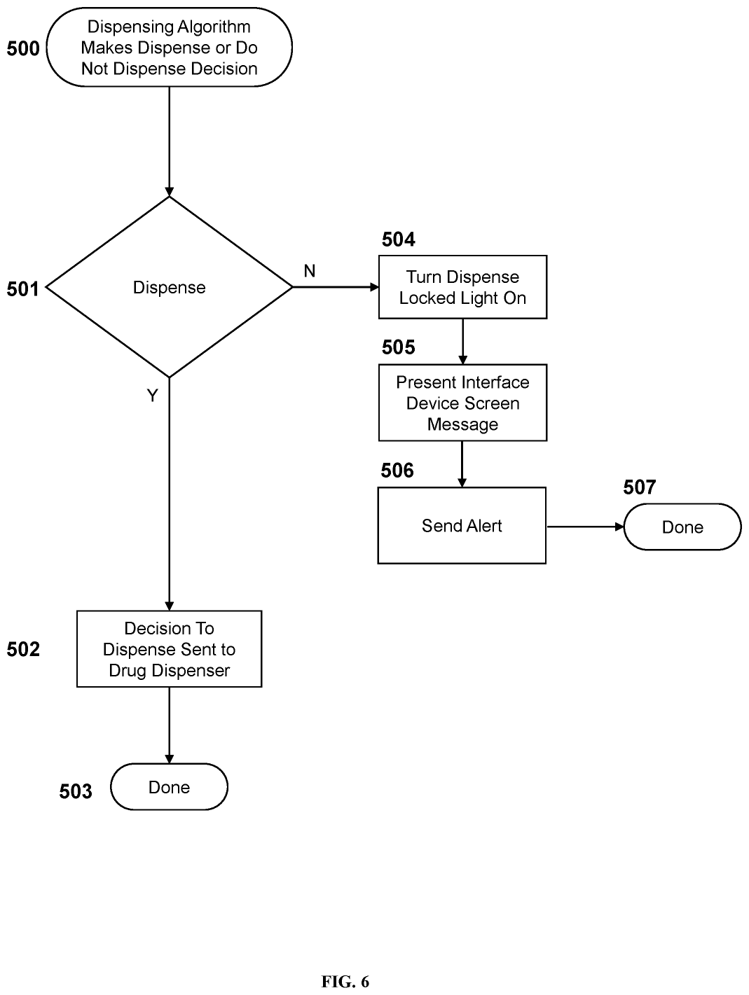

FIG. 6 illustrates an exemplary embodiment of the logic for the Dispensing Algorithm to make a dispense or do not dispense decision 500. The Dispensing Algorithm analyzes the requisite data against defined criteria to make a dispense or do not dispense decision 501. If the decision is not to dispense, the Routine turns on the Drug Dispenser's Locked dispenser light 504, presents a Patient message on the Interface Device with the reason the dose will not be dispensed, when it can be dispensed, or alternatives available to the Patient to get the dose dispensed 505. If the authorized prescribed dose cannot be dispensed, the Routine will also send an alert 506 to designated personnel. Then the Routine is done 507. If the decision is to dispense the dose, the Routine sends an authorization to the Drug Dispenser to dispense the dose 502 at which point the Routine is done 503.

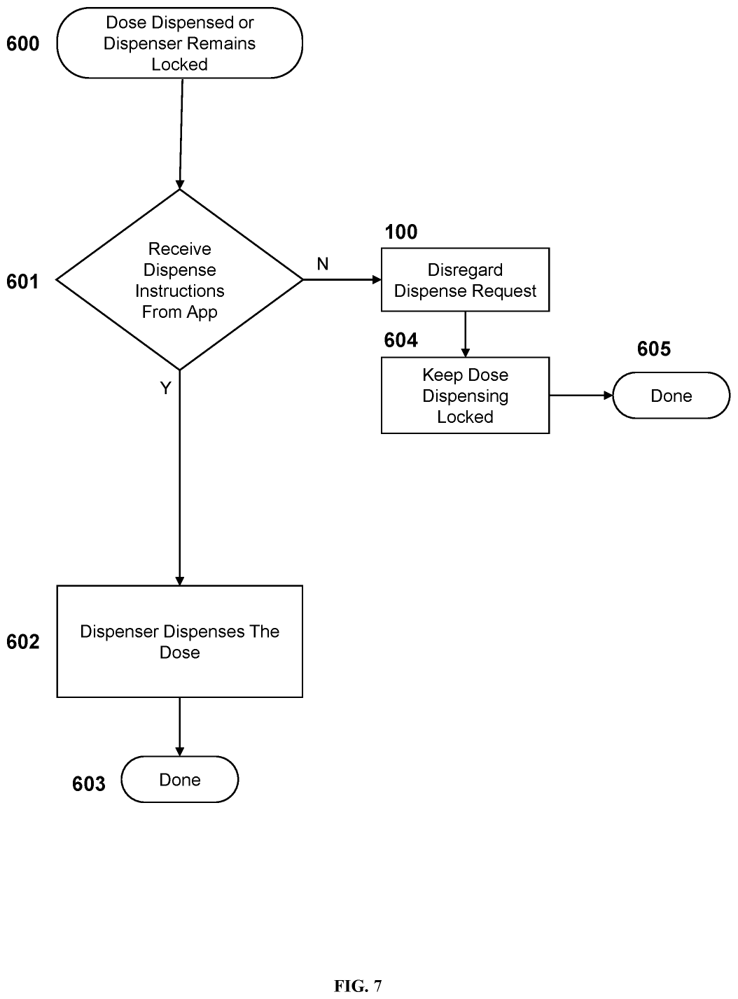

FIG. 7 illustrates an exemplary embodiment of the logic where the Drug Dispenser dispenses the dose or remains Locked 600. If the Drug Dispenser receives a do not dispense instruction from the App 601, it disregards the Drug Dispenser's Dispense Button command 100 and keeps the Drug Dispenser in a Locked position 604 with the Drug Dispenser's Locked light on 504. The Locked light will remain on for a specified period of time after which time the Drug Dispenser's firmware Routine will turn the light off and put the Drug Dispenser in sleep mode at which point the Routine is done 605.

### IV. Examples

The embodiment of the invention can be utilized to, 1) improve the Drug's safety profile by ensuring proper Patient personalized prescribing and Prescription management (e.g., personalized dispensing), 2) ensure the Medication is efficacious for the Patient, 3) preclude dose mediated adverse events, 4) ensure Prescription Compliance, 5) prevent the Patient from inadvertently taking a duplicate dose, 6) enable PRN dosing, and 7) avoid Drug-Drug interactions, In short, to personalize oral Medication therapy by improving the Medication's Drug/safety profile—to provide Point-of-Care Pharmacy type control.

Single-click Dispensing of a Drug dose from a Drug Dispenser enables the transparent delivery of “Point-of-Care Pharmacy that replaces the pill bottle with an App controlled, Single-click Drug Dispenser.

### Antihypertensives

Antihypertensives are a type of Drug used to treat high blood pressure. There are many different types of antihypertensive agents, and they work in different ways to lower blood pressure. Some remove extra fluid and salt from the body. Others relax and widen the blood vessels or slow the heartbeat. A person may respond better and have fewer side effects with one Drug than with another. Some Patients need more than one antihypertensive agent to lower their blood pressure.

Antihypertensive Medications are divided into many different categories, each which works differently and causes different side effects:
Diuretics (thiazide diuretics, potassium-sparing diuretics, loop diuretics, combination diuretics)
Beta-Blockers
Angiotensin Converting Enzyme Inhibitors (ACE)
Angiotensin II Receptor Blockers (ARBs)
Calcium Channel Blockers
Alpha-Blockers
Alpha-Beta-Blockers
Central Agonists
Vasodilators
Aldosterone Receptor Antagonists
Direct Renin Inhibitors
Combination Drugs

### A. Overview

About one of three U.S. adults—or about 75 million people—have high blood pressure. As of 2016, only about half (54%) of these people had their high blood pressure under control. Hypertension is highly prevalent in older age and accounts for a large proportion of cardiovascular (CV) morbidity and mortality worldwide. Clinical trials evidence supports the use of antihypertensive Drugs in hypertensive Patients due to benefits in reducing CV disease and mortality. Hypertension when untreated is associated with increased morbidity and mortality due to long-term complications that include cardiovascular, cerebrovascular and chronic kidney disease and possibly also cognitive decline and/or dementia. Conversely, a consistent J-curve relationship exists between low Diastolic Blood Pressure (DBP) and all-cause death—so it is important to ensure the blood pressure remains within a normal range/the person's target range. Treatment of high blood pressure lasts for life.

Only about a third of people with hypertension reach the blood pressure goal of less than 140/90 mm Hg. One reason is that some doctors do not treat hypertension aggressively enough. In other cases, people do not take their Medication as prescribed or do not adopt the recommended lifestyle changes. Regular visits to the doctor are also an important part of keeping blood pressure under control. People with Stage 1 Hypertension that are otherwise healthy should see their physician once every month until their blood pressure goal is reached. However, people with other health problems or Stage 2 Hypertension should visit their doctor more frequently—typically every two to four weeks. Once the blood pressure has been lowered to the desired level and/or has stabilized, doctor visits can usually be reduced to every three to six months, although people with other health conditions (such as diabetes or heart disease) may need to visit their doctor more often.

Medication Compliance is critical. If a dose is missed or if the dosage amount is split, the person's blood pressure can rise to dangerous levels, putting the person at risk for heart attack, stroke, heart failure and kidney failure.

Some over the counter, prescription Drugs, and supplements can raise blood pressure and/or interfere with the effectiveness of high blood pressure Medications. These Drugs can include: steroids, non-steroidal anti-inflammatory Drugs (NSAIDs), nasal decongestants and other cold remedies, diet pills, cyclosporine, erythropoietin, tricyclic antidepressants and monoamine oxidase inhibitors, etc.

It takes time to find the right dose. Different people can respond very differently to Medications. Everyone has to go through a trial period to find out which Medications work best with the fewest side effects. Time is required to adjust to a Drug. This may take several weeks, but the results will generally yield a better clinical outcome.

Once stabilized, blood pressure should be monitored on a regular basis. A single high reading is not an immediate cause for alarm. If a reading is slightly or moderately higher than normal, blood pressure needs to be measured a few more times. If the reading remains slightly or moderately higher, then the person needs to consult their healthcare professional to verify if there is a health concern or whether there may be an issue(s) with the blood pressure monitor.

If blood pressure readings suddenly exceed 180/120 mm Hg, the person needs to wait five minutes and test again. If the readings are still unusually high, the doctor needs to be contacted immediately. The person is experiencing a hypertensive crisis.

If blood pressure is higher than 180/120 mm Hg and the person is experiencing signs of possible organ damage such as chest pain, shortness of breath, back pain, numbness/weakness, change in vision, difficulty speaking, the person needs to call 9-1-1.

The concerns regarding antihypertensive Drug therapy highlight the importance of primary prevention of hypertension combined with accurate elucidation of the blood pressure (BP) profile. Hypertensive Patients presenting to clinical practice should be routinely screened for orthostatic hypotension; and white coat hypertension should be ruled out. Interventions that lower DBP below 60 mmHg, exacerbations of postural hypotension (aka orthostatic hypotension) and complications associated with polypharmacy contribute to increased risk of falls and/or fractures (in the elderly).

Falls are one of the most common health concerns facing elderly persons today. About one-third of community-dwellers over the age of 65 and nearly one-half of institutionalized persons or persons over the age of 80 will fall each year. Almost half of fallers will experience a repeat fall within the next year. While most falls result in no injury, 31% of falls result in an injury requiring medical attention or restriction of activities for at least one day. Most of these are minor soft tissue injuries, but 10-15% of falls result in fracture, and 5% of falls result in more serious soft tissue injury or head trauma. Among nursing home residents, the incidence of major soft tissue injury or fracture related to a fall is twice that found in community dwelling elderly.

Falls may have other important consequences, even among elders without a fall-related injury. Falls are associated with greater functional decline, social withdrawal, anxiety and depression, and an increased use of medical services. Fear of falling is common among elderly fallers, and fear of falling has been associated with impaired mobility and decreased functional status. As a result, older adults who have fallen are at greater risk of becoming institutionalized regardless of whether they have experienced an injurious fall.

The total cost of fall-related injuries to the U.S. Health care system is substantial. Almost 8% of persons over the age of 70 will seek medical care in the emergency room secondary to a fall-related injury, and about one-third of these persons will be admitted to the hospital. In 2000 the U.S. health care system spent $19 billion on the direct medical costs of fall related injuries. Hip fractures alone, which are commonly associated with falls, cost the U.S. health care system over $8.7 billion per year.

Although fall-related injuries are not a common cause of death in the elderly, accidental falls are the leading cause of unintentional injury deaths in those aged over 65 years. Death related to falls increases with advancing age and greater number of co-morbidities. Certain fall-related injuries, such as hip fractures, are associated with a high mortality within the first six months, particularly in men.

The only cardiac Medications that were associated with falls were diuretics, Type Ia anti-arrhythmic Drugs (e.g. procainamide), and digoxin. The causes of orthostatic hypotension include the following which means a Patient who is already hypotensive may be put at risk if they continue to take their antihypertension Medication:
Aging (orthostatic hypotension is more common in older people)
Hypovolemia (a drop in the volume of blood) and dehydration (low fluid volume in the body). Common causes of these are bleeding, elevated sugar, diarrhea, vomiting, and Medications like thiazide diuretics (HCTZ) and loop diuretics (furosemide, bumetanide)
Dehydration (low fluid volume in the body)
Immobility (for instance, staying in bed for a long time)
Pregnancy
Heart conditions, including heart attack, heart failure, irregular heart rhythm, and valve disease
Anemia (low red blood cell count)
Parkinson's disease
Diseases of the endocrine system, including diabetes, adrenal insufficiency, and thyroid conditions
Medications that are used to treat elevated blood pressure, such as beta blockers, calcium channel blockers, ACE inhibitors, nitrates, and angiotensin II blockers. Patients who are at risk for this are those with conditions listed above (for example, diabetes, Parkinson's)
Other Medications for anxiety, depression, erectile dysfunction, or Parkinson's disease
Substances that are taken at the same time as blood pressure Medications, such as alcohol, barbiturates, and other Medications
Hot weather
   This means that the person needs to talk with their doctor/care giver to ascertain whether to skip the dose and the next steps as care has to be taken before stopping an antihypertensive Medication.

### B. Antihypertensive Single-Click Dose Dispensing

Antihypertensives serve as an excellent exemplary of how the embodiment of the invention can be utilized to prevent dose mediated adverse events and improve Medication management and Patient outcomes.

When the prescription is digitally entered (either as an electronic prescription or entered at the pharmacy from a written prescription) it does two things: 1) instructs the pharmacy to dispense the Medication, and 2) instructs ACCOY's APP Generation Servers to create and send an email with a link to download the App that controls dose dispensing on a Tethered Drug Dispenser.

The Patient then clicks on the respective Android or iOS store link on the email. This takes the Patient to the App in the respective App store where the Patient clicks on the download link and downloads the App onto their smartphone. Clicking on the downloaded App on the smartphone opens the App which requires certain Patient information and authorizations. When completed, the App automatically and securely connects with ACCOY's central servers, in keeping with Patient information security regulations. ACCOY's App generation program then updates the Patient's App on the smartphone with prescription and related information—transforming the App into a Patient personalized Drug and Patient Specific App that controls Dose-by-Dose Dispensing on a tethered dispenser containing the specified Drug (in this case, an antihypertensive). The prescription information cannot be changed by the Patient. It can only be changed by authorized personnel.

The App then has the Patient execute a Handshake with the Patient's Smartwatch to enable secure capture of the values required by the dispensing App. This enables the App to subsequently 1) biometrically authenticate that the data being captured is the Patient's data, and 2) download the data required by the App's dose Dispensing Algorithm to make a dispense, alert and/or do not dispense the dose decision (e.g., Heart Rate, heart rhythm, blood pressure, sweat, accelerometer data to ascertain balance, trips, and falls, etc.).

The App will then periodically access the Smartwatch, based upon a designated schedule between doses and at a designated amount of time before an authorized dose is due, as well as when the dispenser Dispense Button is selected, to capture trended and point in time data that allows the App to ascertain if the Patient's blood pressure is within the target range over a specified timeframe. The time intervals are designated by the Drug and Patient specific App.

The Patient then goes to the pharmacy to pick up their Medication (or receives it in the mail from a mail order pharmacy). The Patient then opens the box containing the dispenser prefilled with the prescribed antihypertensive. One click on the Dispense Button on the Drug Dispenser initiates the Bluetooth (can also be wi-fi, etc.) connection, and once connected, tethers the dispenser to the Drug and Patient specific App. After tethering, dose dispensing can only be controlled by the Drug and Patient specific App. This enables Single-click dose dispensing from the dispenser as authorized by the prescription and the Apps' dose Dispensing Algorithm.

Now the dispenser is ready to dispense.

A Single Click on the Dispense Button on the Drug Dispenser results in dispensing the dose if the dose dispense request is within a specified timeframe of when the dose is due. The App takes data measures right before the authorized dose dispense time as authorized by the prescription, and transmits dose dispensing authority to the dispenser if everything was within an acceptable dispense range. The Single Click also instructs the dispenser to connect with the App, confirm the dose has been dispensed, and to initiate the tethered Smartwatch to take new readings to correlate with the dose dispense time.

If the App ascertains a dose should not be dispensed, it will keep the dispenser Locked and alert the Patient why the dose cannot be dispensed, when the dose will be available, and the steps the Patient should take if the dose will not be available. If the issue is one which may be addressed by medical personnel, the Patient can click on the connect button on the alert page on the smartphone to be connected to ACCOY's 24/7 365 medically staffed support center. The support personnel have tools to ascertain whether or not the dose should be dispensed, and if the decision is that it should be, the support center has the ability to remotely override the lock and dispense the dose.

If the App ascertains to dose cannot be dispensed and the Patient does not elect to contact the support center within a designated timeframe, the App will send Patient authorized and security regulation compliant alerts to specified care givers, family, etc. alerting them the dose could not be dispensed and what actions the Patient should take.

A number of actions take place when a Patient initiates the Single-click dispense sequence. The App's dispense Algorithm checks to see if the dispense request is authorized by the prescription. It checks if the Patient's values are within the normal range. It uses data analytics to ascertain if the Patient may have missed a dose or if the Patient may be splitting Medication as shown by trended value aberrations. It checks the blood pressure reading to check for issues of concern, such as suddenly high readings that may indicate a hypertensive crisis or change in values that indicate a change in Medications or lifestyle. Check concomitant Medications for potential Drug interactions. Correlate activity, hydration, and diet information with the Medication and the trended blood pressure to assist the Prescriber in managing the Patient's blood pressure. Check trended and the latest value to identify hypotension. Evaluate Patient balance, trip, and fall data against trended blood pressure and other data to ascertain if the Patient may be at risk of hypotension induced falls.

The App uses the data to ascertain: 1) if it should take additional value readings to ascertain if an identified potential problem is of concern, 2) if the dose should not be dispensed, and 3) if Patient and/or caregiver alerts are warranted.

In this way the Single-click Dispensing is really Single-click “controlled” Point-of-Care Pharmacy dispensing because it transparently:
Assists the Prescriber in ascertaining if the antihypertensive is effective in lowering the Patient's hypertension.
Monitor's the Patient's blood pressure throughout the day, whether awake or asleep, whether resting, working, or exercising—providing a true picture of the Patient's blood pressure—enabling better Medication/blood pressure management.
Assists in dose titration.
Screens for possible concomitant Drug interactions.
Monitors, on a real-time basis, blood pressure control.
Assists in the identification of lifestyle, exercise and diet on the Patient's blood pressure.
Alerts Prescribers if something has changed that is affecting the antihypertensive's ability to lower the Patient's blood pressure (versus prior efficacy).
Alerts the Patient and caregivers if the Patient is having a hypertensive crisis.
Identifies hypotension.
For certain antihypertensives, e.g., diuretics, digoxin, etc., prevents falls by precluding dispensing a dose that will make the Patient hypotensive and prone to falling.
   Furthermore, it allows the natural substitution of a pill bottle for a smart, App-controlled Drug Dispenser that transparently provides better Medication and Patient management.

### Opioids

Opioids serve as another excellent exemplary of how the embodiment of the invention can be utilized to improve Medications' management and Patient outcomes.

### A. Background

The following describes some, but not all, of the key opioid side effects as background for the subsequent opioid examples of the embodiment:

Cognitive Impairment—It is well known that larger doses of opioids are markedly impairing, leading to drowsiness, lethargy, and even death. At least one prospective study has demonstrated that those with chronic pain on opioid therapy have cognitive deficits including reduced spatial memory capacity and impaired performance in working memory assessment (Schiltenwolf et al., 2014).

Respiratory depression—Opioids adversely affect the respiratory system. Carbon dioxide (CO₂) levels in the blood stimulate our respiratory drive. As breathing slows down, CO₂ levels increase, which stimulates the brainstem to increase the respiratory rate.

Low oxygen levels do not stimulate breathing so sensitivity to CO₂ levels is an important function of nerve cells in the brainstem. Opioids block that feedback loop. When an individual overdoses on an opioid, the high levels of opioid will decrease alertness and induce sleep. During sleep, it is the CO₂ feedback loop that keeps people breathing. However, when blocked by the high levels of an opioid, breathing slows or stops and the person who has overdosed literally suffocates.

Heart Rate—Heart Rate may become either rapid or very slow. Some opioid users may also develop postural hypotension or a severe fall in blood pressure on standing up from a sitting or lying position.

This is also problematic for individuals with lung disease or sleep apnea. People with chronic lung disease often need elevated carbon dioxide levels to stimulate them to breathe more deeply. Taking opioids will blunt this response, causing people with lung disease to breathe slower and therefore have low oxygen levels.

Sleep apnea is similar as people periodically stop breathing at night until their carbon dioxide levels get high enough to stimulate their brain to signal them to gasp for breath. When opioids interfere with this response the effect can be life threatening. Opioids have been shown to worsen the apnea episodes in those with sleep apnea (Jungquist, Flannery, Perlis, & Grace, 2012).

Miosis—Opioid use results in the formation of small, constricted pupils, similar to how pupils respond to bright light.

Constipation—Opioids cause sluggish peristaltic movements in the digestive tract. This causes stasis or loss of movement of the intestinal contents and leads to severe constipation, especially in the case of long-term use.

Drowsiness or Sedation—Opioids, and in particular morphine, are known to cause severe sedation and drowsiness.

Myoclonus—High doses of opioids can result in muscle rigidity and abnormal movement of the limbs and muscles.

Hyperalgesia—Opioid-induced hyperalgesia (OIH) is another side effect of the use of opioids. Opioid hyperalgesia is a phenomenon where the body develops an increased sensitivity to pain secondary to opioid use (hyper—over or excess, algesia—sensitivity to pain).

Pain is an important part of our body's defense system, warning us of current or impending damage or injury. As opioids decrease our brain's sensitivity to pain signals coming from the rest of the body, our brain begins to compensate by increasing our recognition of and sensitivity to pain. The pain neurons going to the brain actually change to make them more responsive to pain and increase our perception of pain. This change is called neuroplasticity of the nerve cell. Many mechanisms are believed to be involved in these changes (M. Lee, Silverman, Hansen, Patel, & Manchikanti, 2011). The result of this change is that after opioid levels decrease, our pain fibers are more sensitive than before consuming the opioid which results in an increase in pain.

Unfortunately, increasing pain can also mean disease progression or the development of tolerance to the current opioid dose. For these conditions, opioid doses are usually increased. In contrast, the treatment of opioid hyperalgesia is the decrease or discontinuation of opioids.

Tolerance and withdrawal—Tolerance occurs when the body has developed physiologic (both neuroplastic and chemical) changes that result in decreased effectiveness of the medicine necessitating a higher dose to get the same effect. Withdrawal is the unpleasant symptoms that develop upon decrease or discontinuation of that medicine. Brain changes and measurable withdrawal symptoms can occur after one dose of opioid (Rothwell, Thomas, & Gewirtz, 2012).

In clinical practice, withdrawal symptoms can occur after five to seven days of opioid Medication (Anand et al., 2010). Withdrawal symptoms may consist of myalgia (muscle pain), chills, sweats, anxiety, increased pain, rapid heartbeat, dilated pupils, yawning, diarrhea and nausea.

Withdrawal is extremely unpleasant and can be relieved by taking another opioid dose. Tolerance and withdrawal are not considered addiction. Tolerance occurs because of the physiologic changes resulting from exposure to opioids. Withdrawal is the unpleasant physical and emotional symptoms that occur upon withdrawal of the opioid after tolerance has developed.

Addiction—Addiction is characterized by inability to consistently abstain, impairment in behavioral control, craving, diminished recognition of significant problems with one's behaviors and interpersonal relationships, and a dysfunctional emotional response.

Benzodiazepines—It is also important to realize that the risk of death is markedly increased when opioids are taken with benzodiazepine Medications. The combination of opioids and benzodiazepines are the leading cause of overdose deaths when multiple Medications are involved (Calcaterra, Glanz, & Binswanger, 2013). Despite this danger, benzodiazepines are prescribed to about 30% of people on chronic opioid therapy (Nowak, Abou-Nader, & Stettin, 2014).

### B. Opioids Single-Click Dose Dispensing

Point of care opioids Single-click Dose Dispensing can be utilized for opioids Medication and pain management. The Opioid App utilizes one or more biometric values to validate that the data that will be utilized for the Dispensing Algorithm are in fact the Patient's values (using things like heart rhythm, blood pressure, respiration, voice print, etc.). These values can be automatically taken at designated intervals between and just before authorizing a prescribed dose as well as at the point when the Patient uses Single-click Dose Dispensing of the opioid from the Drug Dispenser.

The required Algorithm inputs can be automatically aggregated by the smartphone and/or by a tethered device, such as a Smartwatch and/or wearable sensor. The data capture can be trended data or single point in time data or a combination thereof.

Single-click Dose Dispensing can be utilize to preclude dispensing a dose that may cause a dose mediated adverse event on an exception basis or may be utilized between doses which require Patient reported inputs in order to facilitate dose dispensing and/or to ensure Patient access to the Medication in the advent of a smartphone failure.

As an example, the App can monitor and trend the Patient's respiratory rate to ascertain if the Patient is or may overdose if they take the dose. Conversely, the App may monitor the Patient's Heart Rate, respiratory rate, speech pattern, gate, balance, sleep, etc. on an ongoing basis. The trended data is utilized by the Dispensing Algorithm to ascertain if the Patient may be at risk of overdosing if the Patient takes the dose.

The objective of Single-click Dose Dispensing for opioids, in this exemplary, is ensuring Patient prescription Compliance, preventing misuse, and preventing a dose mediated adverse event.

Trended data analytics and multifactorial analysis can be utilized to identify outliers from trended norms to identify misuse and potential Abuse.

Continual, passive 24/7 365 Patient value aggregation can also be utilized to identify individuals falling into respiratory distress and automatically alert Appropriate personnel in keeping with Appropriate Patient data regulations.

Because passive measurements used to ascertain if an opioid Patient may overdose if they take the opioid dose require trended measurements, e.g., respiration rate, Heart Rate, etc., the data values must be automatically taken before the authorized dose dispensing timeframe. Otherwise, Single-click Dose Dispensing requires a delay between the time the Patient activates the Dispense Button and when the dose is dispensed.

What is claimed:

1. A method of instructing a Drug Dispenser to dispense a Drug dose comprising: a Drug Dispenser under the control of a Drug dose dispensing routine; and in response to only a single action being performed, sending a request to dispense the Drug dose along with a Patient identifier and a Drug Dispenser identifier to the dispensing control App; under control of a single-action dispensing component of the App, the App receiving the request; retrieving additional information previously stored for the Patient identified by the identifier in the received request; requesting current Patient data from one or more devices with sensors and/or Patient diaries to aggregate Patient data; using a Drug Dispensing Algorithm to evaluate said stored and retrieved Patient data to make a dispense or do not dispense the dose decision, Appending the new Patient data to current data sets, and dispensing the dose and recording when the dose was dispensed, or recording why and the time the dose was not dispensed; and thereafter updating the App stored information.

2. The method of claim 1 wherein the displaying of information includes displaying information indicating that Single action dispensing is available.

3. The method of claim 1 wherein the single action is clicking, tapping, and/or touching a dispense button.

4. The method of claim 1 wherein the single action is Speaking of a Sound.

5. The method of claim 1 wherein the single action is utilizing a biometric log to initiate the single action.

6. A dispenser client system routine for dispensing a Drug dose comprising: an identifier that identifies a Drug Dispenser; an identifier that identifies the Patient, a label component for displaying information identifying the Drug; a Single-action ordering component that in response to performance of only a Single action, sends a request to the App to dispense the Drug, the request including identifiers so that the App can locate additional information needed to make a dispense the Drug dose or do not dispense the Drug dose decision, that the Drug Dispenser can dispense the dose, and update related dispenser routine and App data repositories that the dose was dispensed or that the dose was not dispensed and why.

7. The dispenser client system of claim 6 wherein the Drug Dispenser identifier is a Drug Dispenser Serial Number.

8. The dispenser client system of claim 6 wherein the Patient identifier is any one or a combination of: the ACCOY electronic biometric identifier used by the App, a medical record identifier, the Patient's health insurance identifier, etc.

9. The dispenser client system of claim 6 wherein the label component for identifying the Drug is any one or a combination of a label adhered to the Drug Dispenser and/or an electronic display on the Drug Dispenser.

10. The dispenser client system of claim 6 wherein the Single action is the physical or voice activation of the Dispense Button on the Drug Dispenser.

11. An App System for generating an order for the Drug Dispenser to dispense a Medication dose comprised of an App Controlled Drug Dispenser with a Single-action dispensing component including: a receiving component for receiving requests from the Patient to dispense the dose, the request being Sent in response to only a single action being performed; and a dispense authorization component that retrieves Patient data from the data Storage medium; a data gathering component to retrieve Patient data from defined sensors; and a Dispensing Algorithm that utilizes the gathered Patient data to make a dispense the dose or do not dispense the dose decision; and the dispense the dose component that in keeping with the Single-action dispense dispenses the dose.

12. The App System of claim 11 wherein the request is Sent by a client System routine in response to a single action being performed.

13. The App System of claim 11 for ordering the dispensing of a Medication dose using a client System, wherein the App system uses an identifier Sent along with the request to identify additional information needed to dispense the dose.

14. The method of claim 11 wherein the identifier identifies the Drug Dispenser and the App System provides the identifier to the Drug Dispenser.

15. The method of claim 11 wherein the Drug Dispenser system and the App System communicate Via Bluetooth or similar close proximity communications protocol.

16. The method of claim 11 wherein the single action is touching and/or tapping the Dispense Button on the Drug Dispenser.

17. The method of claim 11 wherein the single action is depressing of the Dispense Button on the Drug Dispenser.

18. The method of claim 11 wherein the single action is using a biometrically activated Dispense Button on a Drug Dispenser.

19. The method of claim 11 wherein the single action is a verbal command for the Drug Dispenser to dispense the Medication.
